(19) United States
(12) Patent Application Publication
(10) Pub. No.: US 2022/0020454 A1
(43) Pub. Date: Jan. 20, 2022

(54) METHOD FOR DATA PROCESSING TO DERIVE NEW DRUG CANDIDATE SUBSTANCE

(71) Applicant: MEDIRITA, Seoul (KR)

(72) Inventors: Young Woo PAE, Seoul (KR); Seung-Hyun JIN, Seoul (KR)

(73) Assignee: MEDIRITA, Seoul (KR)

(21) Appl. No.: 17/428,619

(22) PCT Filed: Dec. 16, 2019
(86) PCT No.: PCT/KR2019/017793

§ 371 Date: Aug. 4, 2021

(30) Foreign Application Priority Data
Mar. 13, 2019 (KR) 10-2019-0028788
Mar. 13, 2019 (KR) 10-2019-0028789
Mar. 13, 2019 (KR) PCT/KR2019/002918
Mar. 13, 2019 (KR) PCT/KR2019/002919
Dec. 10, 2019 (KR) 10-2019-0163398

(51) Int. Cl. G16C 20/70; G16H 70/40; G16H 20/10; G06F 16/901; G16H 50/70; G16C 20/40

(52) U.S. Cl. CPC G16C 20/70; G16H 70/40; G16C 20/40; G06F 16/9024; G16H 50/70; G16H 20/10

(57) ABSTRACT

A method includes generating a DB matrix composed of a selected biological entity and a selected type of mutual association degree from an omics DB, receiving a search word, extracting biological entities, extracting a degree of mutual association between the search word and the biological entities from the DB matrix, generating a first knowledge network in which the search word and each of the biological entities are used as nodes and a plurality of nodes are connected using a connection line according to a degree of mutual association between the search word and the biological entities or a degree of mutual association between the biological entities, computing a graph theory index for each of the plurality of nodes of the first knowledge network, and generating a second knowledge network using some nodes selected using the graph theory index among the plurality of nodes of the first knowledge network.

### TECHNICAL FIELD

The present invention relates to a method for developing a new drug, and more particularly, to a method for data processing to derive a new drug candidate substance from an omics database.

### BACKGROUND ART

It is known that it takes a total of 15 years and costs 2 to 3 trillion won on average to develop a new drug. Most of all, it is known that it takes about 6 years to discover the new drug candidate substance before preclinical trial.

In general, in order to discover the new drug candidate substance, which is a first stage in a pipeline for developing the new drug, a large number of specialized research personnel are going through a process of searching for huge amounts of information one by one and inferring association between major biological entities from this search.

Meanwhile, according to the Life Intelligence Consortium (2017) recently launched in Japan, when artificial intelligence technology is used to develop the new drug, it is predicted that the time required to develop a new drug can be reduced to about 40% and the cost can be reduced to about 50%.

### DISCLOSURE OF THE INVENTION

### Technical Problem

A technical problem to be solved by the present invention is to provide a method for data processing to discover a new drug candidate substance. Another technical problem to be solved by the present invention relates to a method for generating a multiomics network having a hierarchical structure from a human omics database (DB) and generating a refined knowledge network from the multiomics network.

### Advantageous Effects

Refined information on biological entities related to a predetermined search word and a degree of mutual association between the biological entities can be extracted within a short time without searching for huge amounts of information one by one in order to discover a new drug candidate substance. Accordingly, it is possible to significantly reduce the cost and period required to discover a new drug candidate substance or a target of new drug candidate substance.

### BRIEF DESCRIPTION OF THE DRAWINGS

FIG. 1 is a block diagram of an apparatus for processing data for discovering a new drug candidate substance, according to an embodiment;

FIG. 2 illustrates a flowchart of a method for data processing to discover a new drug candidate substance by the apparatus for processing data, according to an embodiment;

FIG. 3 illustrates a search word input, according to an embodiment;

FIG. 4 illustrates a DB matrix generated in step S205, according to an embodiment;

FIG. 5 illustrates a DB matrix generated in step S205, according to an embodiment;

FIG. 6 is a first knowledge network according to an embodiment;

FIG. 7 illustrates the classification of types of hubs according to a participation coefficient (PC), according to an embodiment;

FIG. 8 is a second knowledge network generated from a search word “epilepsy syndrome”, according to an embodiment;

FIG. 9 illustrates an example in which an omics level (biological entity) is input, according to an embodiment;

FIG. 10 illustrates an example in which a type of mutual association degree is input, according to an embodiment;

FIG. 11 is a block diagram of an apparatus for processing data for discovering a new drug candidate substance, according to an additional embodiment;

FIG. 12 illustrates a flowchart of a method for data processing to discover a new drug candidate substance by the apparatus for processing data, according to an additional embodiment; and

FIG. 13 illustrates a flowchart of how the apparatus for processing data searches for a drug-possible path according to an embodiment.

### BEST MODE FOR CARRYING OUT THE INVENTION

A method for data processing to discover a new drug candidate substance performed by an apparatus for processing data, includes generating a DB matrix composed of a selected biological entity and a selected type of mutual association degree from an omics DB, receiving a search word, extracting biological entities that belong to an omics level different from the search word and are related to the search word from the DB matrix, extracting a degree of mutual association between the search word and the biological entities from the DB matrix, generating a first knowledge network in which the search word and each of the biological entities are used as nodes and a plurality of nodes are connected using a connection line according to a degree of mutual association between the search word and the biological entities or a degree of mutual association between the biological entities, computing a graph theory index for each of the plurality of nodes of the first knowledge network, and generating a second knowledge network using some nodes selected using the graph theory index among the plurality of nodes of the first knowledge network, in which the search word includes at least one of a gene name, a protein name, a metabolite name, a symptom name, a disease name, a compound name, and a drug name, the biological entities include at least one of genes, proteins, metabolites, symptoms, diseases, compounds, and drugs, categories of the degree of mutual association include participate, covariate, regulate, associate, bind, upregulate, resemble, treat, downregulate, palliate, include, and express, the graph theory index includes at least one of a shortest path between nodes, a clustering coefficient for each node, and a centrality coefficient for each node, for at least one of the plurality of nodes constituting the first knowledge network, a weight of the connection line is set differently according to the category of the degree of mutual association indicated by the connection line, and the shortest path between nodes is calculated by reflecting the set weight, in the generating of the second knowledge network, the second knowledge network is generated by computing a standard score for at least one of the shortest path between nodes, the clustering coefficient for each node, and the centrality coefficient for each node and deleting a node whose standard score is less than a threshold value and a connection line of the node whose standard score is less than the threshold value, the standard score is a value obtained by dividing a difference between an index value of a predetermined graph theory index for each node constituting the first knowledge network and an average index value of the graph theory indexes for the plurality of nodes constituting the first knowledge network by a standard error, and the DB matrix is generated such that the selected biological entities are arranged on a horizontal axis and a vertical axis, respectively, and the type of mutual association degree is displayed at a point where the horizontal axis and the vertical axis intersect.

The generating of the second knowledge network may include computing the standard score for each of the nodes of the first knowledge network after randomly shuffling all the connection lines constituting the first knowledge network, and the number of times of randomly shuffling may be 1000 times or more.

The generating of the second knowledge network may further include deleting a node having one connection line from among the nodes constituting the first knowledge network and deleting a node having a clustering coefficient of 0 from among the nodes constituting the first knowledge network.

The categories of the degree of mutual association may further include at least one of interact, cause, present, and localize.

Extracting a drug-possible path from the second knowledge network may be further included, and the extracting of the drug-possible path may include selecting drug-disease node pairs whose standard score of a degree of proximity to each of the drug-disease nodes existing in the second knowledge network is less than a reference value, extracting, from among paths for the selected drug-disease node pairs, paths in which the number of intermediate nodes existing in each of the paths is equal to or greater than a reference number, and extracting, as the drug-possible path, a path in which a total sum of centrality coefficients of intermediate nodes of the extracted paths is equal to or greater than a reference value, from among the extracted paths.

A recording medium having recorded therein a program for causing the method for data processing to be executed by a computer may be provided.

### MODE FOR CARRYING OUT THE INVENTION

In the description below, several embodiments will be described clearly and in detail with reference to the accompanying drawings so that those with ordinary knowledge in the art to which the present invention pertains (hereinafter, referred to as those skilled in the art) can easily embody the present invention.

In addition, the term “˜ unit” as used in this specification can mean a hardware component or circuit, such as a field programmable gate array (FPGA) or application specific integrated circuit (ASIC).

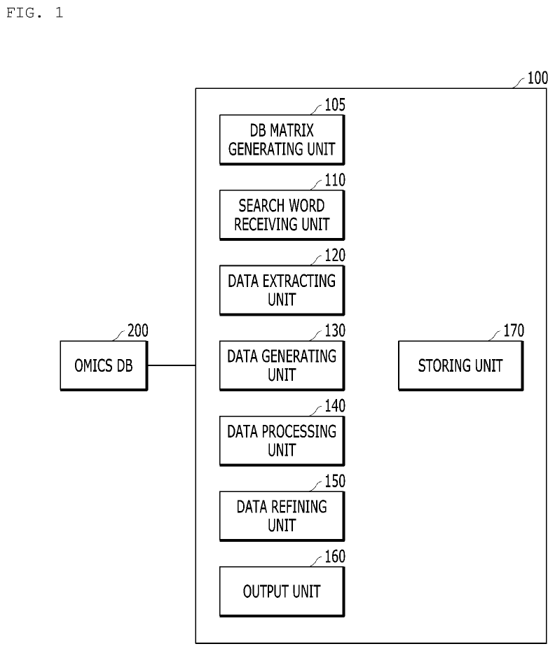

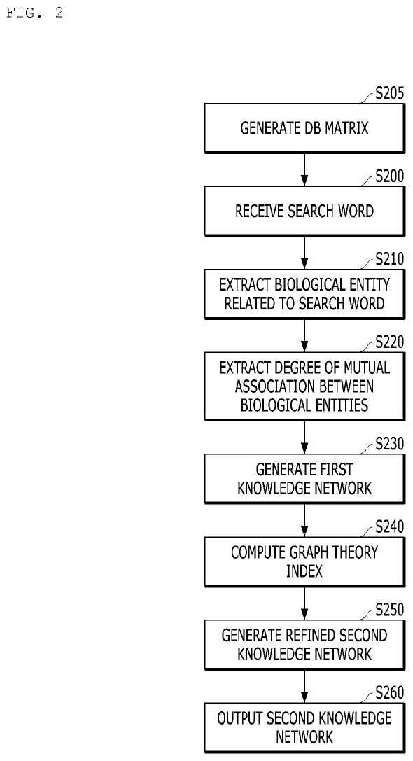

FIG. 1 is a block diagram of an apparatus for processing data for discovering a new drug candidate substance according to an embodiment, and FIG. 2 illustrates a flowchart of a method for data processing to discover a new drug candidate substance by the apparatus for processing data according to an embodiment.

Referring to FIG. 1, an apparatus for processing data 100 for discovering a new drug candidate substance can include a DB matrix generating unit 105, a search word receiving unit 110, a data extracting unit 120, a data generating unit 130, a data processing unit 140, and a data refining unit 150, an output unit 160, and a storing unit 170. The apparatus for processing data 100 can include at least one computing device. For example, the apparatus for processing data 100 can include at least one processor and at least one memory.

Referring to FIGS. 1 and 2, the DB matrix generating unit 105 can generate a DB matrix composed of a DB about at least some omics levels (biological entities) and a DB about at least some types of degrees of mutual associations from an omics DB 200 (S205). The omics levels (biological entities) and the types of degrees of mutual associations for generating the DB matrix can be selected by the user. To this end, the DB matrix generating unit 105 can receive an omics level (biological entity) of at least some of the plurality of levels constituting the omics and receive at least some types degrees of mutual associations among a plurality of types of degrees of mutual associations constituting the omics, in order to generate the DB matrix.

Omics is also referred to as somatics, e.g., there are genetics, transcriptomes, proteomics, metabolomics, epigenetics, lipidomics, etc., and in detail, contents related to anatomy, biological processes, pathways, pharmacological class, symptoms, diseases, compounds, drugs, side effects, etc. can be included, but are not limited thereto. The plurality of omics levels can include a gene level, a transcription level, a protein level, a metabolite level, an epigenetic level, a lipid level, an anatomy level, a biological process level, a pathway level, a pharmacological class level, a symptom level, a disease level, a compound level, a drug level, and a side effect level, etc., but are not limited thereto. Here, the anatomy can mean a tissue, an organ, etc., and the biological process is a series of events including cellular components such as location at the level of the structure in cells, and molecular functions extracted from gene ontology, and the pharmacological class can be a pharmacological effect and a mechanism of action.

The plurality types of mutual association degrees can include “interact”, “participate”, “covariate”, “regulate”, “associate”, “bind”, “upregulate”, “cause”, “resemble”, “treat”, “downregulate”, “palliate”, “present”, “localize”, “include”, “express”, “decrease”, “increase”, etc., and an identification number or an identification symbol can be arbitrarily assigned to each type. The identification number or identification symbol for each type can be set by a user or can be automatically set.

The omics DB 200 can be a big data DB, can be a DB outside the apparatus for processing data 100 according to an embodiment of the present invention, and can be a global public DB that anyone can access or an authenticated person can access under predetermined conditions. The omics DB 200 can store information about an omics level (biological entity) and information about the degree of mutual association between biological entities within the omics level in advance. For example, the omics DB can include a DB for each omics level and a DB for each type of mutual association degree.

The DB for each omics level can include, e.g., a gene DB, a transcription DB, a protein DB, a metabolite DB, an epigenetic DB, a lipid DB, an anatomy DB, a biological process DB, a pathway DB, a symptom DB, a disease DB, a compound DB, a drug DB, and a side effect DB.

The DB for each type of mutual association degree can include an interaction DB, a participate DB, a covariate DB, a regulate DB, an associate DB, a bind DB, and an upregulate DB, a cause DB, a resemble DB, a treat DB, a downregulate DB, a palliate DB, a present DB, a localize DB, an include DB, and an express DB, a decrease DB, and an increase DB. These DBs can be managed and operated by being integrated into one big data DB, or managed and operated by being distributed.

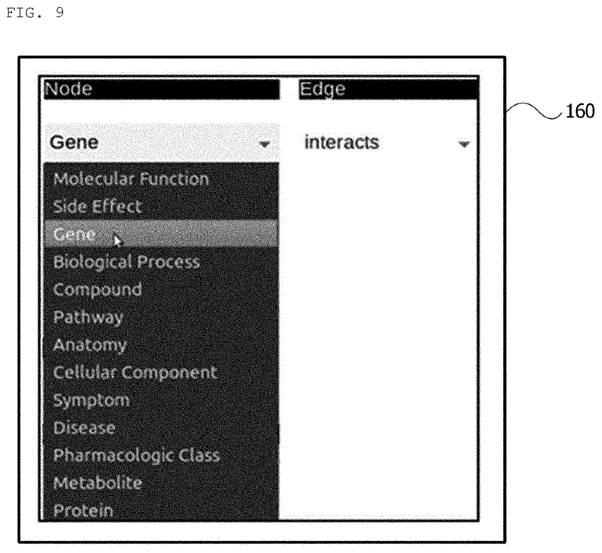

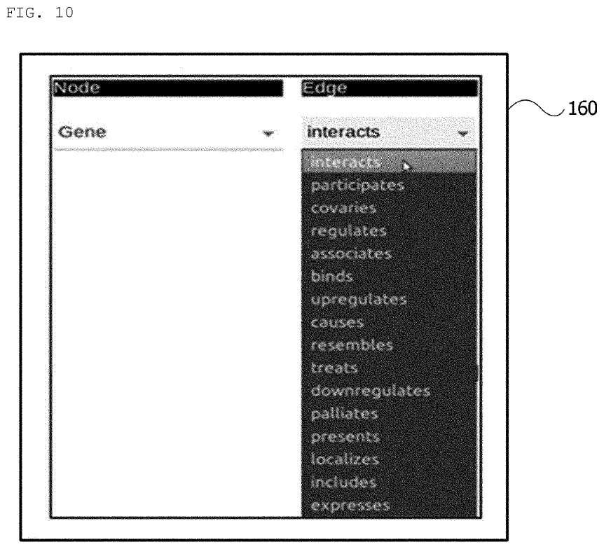

FIG. 9 illustrates an example in which an omics level (biological entity) is input in order to generate the DB matrix according to an embodiment and FIG. 10 is an example in which a type of mutual association degree is input in order to generate the DB matrix according to an embodiment. Referring to FIG. 9, a screen from which a plurality of omics levels can be selected can be exposed through the output unit 160, and at least some of the omics levels can be selected through a user interface from among the plurality of omics levels. Referring to FIG. 10, a screen from which a plurality of types of mutual association degrees can be selected can be exposed through the output unit 160, and at least some of the types of mutual association degrees can be selected through a user interface from among the plurality of types of mutual association degree.

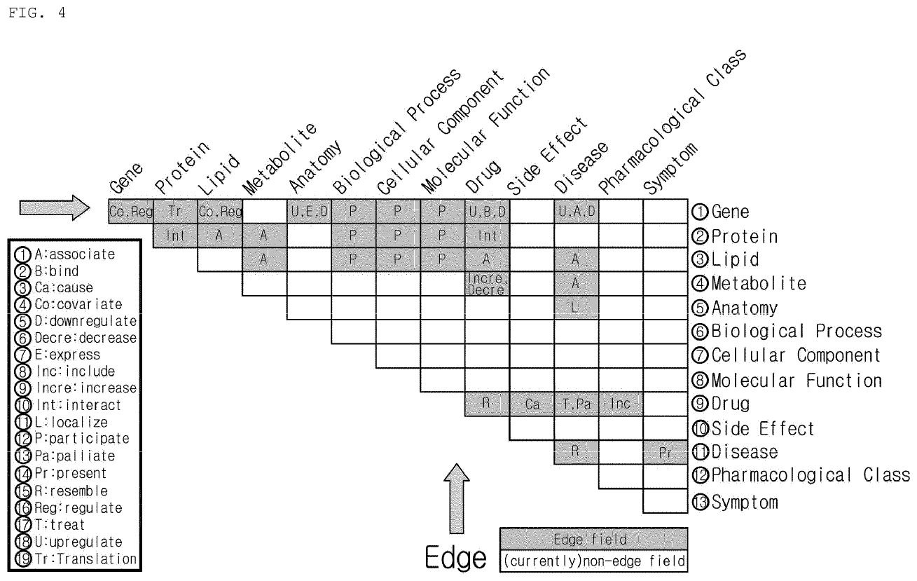

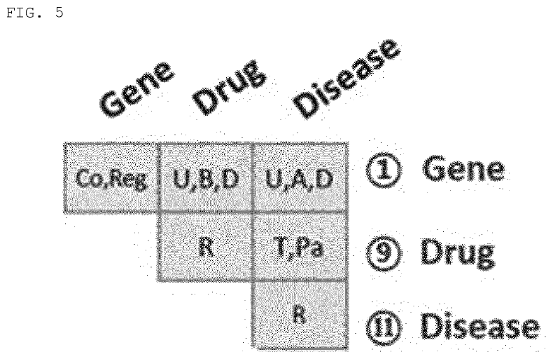

FIGS. 4 and 5 illustrate examples of the DB matrix. If the user selects all the omics levels (biological entities) and all the types of mutual association degrees of the omics DB to generate the DB matrix, the DB matrix can be generated as illustrated in FIG. 4. Referring to FIG. 4, the selected omics levels are disposed on each of a horizontal axis and a vertical axis, and the selected types of mutual association degrees can be generated to be displayed at a point where the horizontal and vertical axes intersect.

For example, a gene level, a protein level, a lipid level, a metabolite level, an anatomy level, a biological process level, a cellular component level, a molecular function level, a drug level, a side effect level, a disease level, a pharmacological class level, and a symptom level can be disposed on each of the horizontal axis and vertical axes of the first matrix, and, at the point where the horizontal axis and the vertical axis intersect, at least one of interact Int, participate P, covariate Co, regulate Reg, associate A, bind B, upregulate U, cause Ca, resemble R, treat T, downregulate D, palliate Pa, present, Pr, localize L, include Inc, and decrease Decre, increase Incre, translation Tr, and express E, which are the types of mutual association degrees, can be displayed.

If the user selects the DB type as the gene level, drug level, and disease level and selects the type of mutual association between DBs as covariate Co, regulate Reg, upregulate U, bind B, downregulate D, associate A, resemble R, treat T, and palliate Pa in order to generate a DB matrix, the DB matrix can be generated as illustrated in FIG. 5.

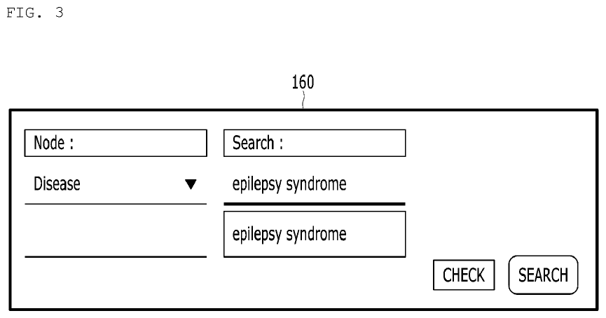

Referring back to FIGS. 1 and 2, the search word receiving unit 110 can receive a predetermined search word (S200). The predetermined search word can be input through the user interface, and can include at least one of a gene name, a protein name, a metabolite name, a symptom name, a disease name, a compound name, and a drug name. For example, the user can input a drug called Bupropion as the search word or a disease called epilepsy syndrome as the search word through the search word receiving unit 110. FIG. 3 illustrates an example in which the predetermined search word is input. Referring to FIG. 3, a screen for inputting the predetermined search word can be exposed through the output unit 160, and the predetermined search word can be input through the user interface. FIG. 3 illustrates an example in which a disease name is selected as a category and epilepsy syndrome is input as the predetermined search word.

Next, the data extracting unit 120 can extract at least one biological entity related to the predetermined search word received in step S200 using the generated DB matrix (S210) and extract a degree of mutual association between the predetermined search word and the extracted biological entity using the generated DB matrix (S220). Here, the biological entity can include at least one of genes, proteins, metabolites, symptoms, diseases, compounds, and drugs, and a level to which the predetermined search word belongs may be the same as or different from a level to which the biological entity belongs. For example, as illustrated in FIG. 3, when the predetermined search word is epilepsy syndrome, which is a disease name, the biological entities extracted in step S210 can include at least one of genes associated with epilepsy syndrome, proteins associated with epilepsy syndrome, metabolites associated with epilepsy syndrome, symptoms associated with epilepsy syndrome, diseases associated with epilepsy syndrome, compounds associated with epilepsy syndrome, and drugs associated with epilepsy syndrome. In addition, the biological entities extracted in step S210 may include a plurality of biological entities for each level. As illustrated in FIG. 3, when the predetermined search word is epilepsy syndrome, which is a disease name, the biological entities extracted in step S210 may include at least one of a plurality of genes associated with epilepsy syndrome, a plurality of proteins associated with epilepsy syndrome, a plurality of metabolites associated with epilepsy syndrome, a plurality of symptoms associated with epilepsy syndrome, a plurality of diseases associated with epilepsy syndrome, a plurality of compounds associated with epilepsy syndrome, and a plurality of drugs associated with epilepsy syndrome.

As described above, when a biological entity associated with a predetermined search term and a degree of mutual association are extracted using the DB matrix in steps S210 and S220, an amount of DB to be searched can be significantly reduced, and accordingly, it is possible to reduce the time and cost for searching for information and extract only information desired by the user.

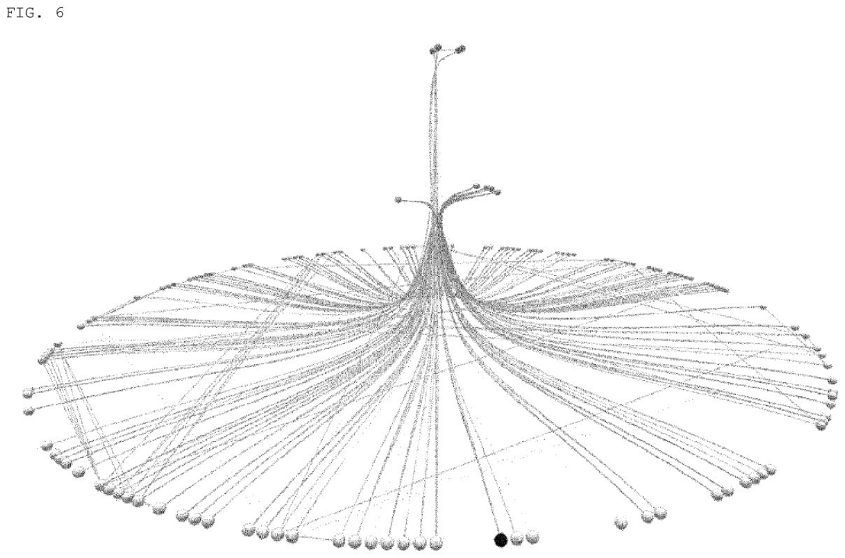

Next, the data generating unit 130 can generate a first knowledge network using the results extracted in steps S210 and S220 (S230). FIG. 6 illustrates an example of a first knowledge network generated according to an embodiment. A circle shape can represent a node, and a line can represent a connection line (edge). Here, the first knowledge network may have a graph form in which the predetermined search word received in step S200 and each of at least one biological entity extracted in step S210 are used as nodes, and a plurality of nodes are connected using connection lines according to the degrees of mutual associations between the predetermined search word and the biological entities extracted in step S220 or the degrees of mutual associations between the biological entities. Nodes within the same omics level can be connected through the connection lines, and nodes within different omics levels can be connected through the connection lines. There can be various paths from node A, which is one of nodes in the first knowledge network, to node B, which is the other one thereof, and all the possible paths can be connected by the connection lines. Here, the knowledge network is a network composed of the degrees of mutual associations between the biological entities, and can also be referred to as a biological network.

Next, the data processing unit 140 can compute the graph theory indexes of the first knowledge network generated in step S230 (S240). According to an embodiment, the graph theory indexes can include at least one of a shortest path between nodes, a clustering coefficient for each node, a centrality coefficient for each node, and a hub characteristic, for each node for a plurality of nodes constituting the first knowledge network.

The shortest path between nodes can mean the shortest path among a large number of paths directing from node A to node B in the first knowledge network. Hereinafter, a method for calculating the shortest path between node A, which is one of the biological entities, and node B, which is the other of the biological entities, will be described.

There are various paths directing from node A to node B, and node A and node B can be directly connected, or at least one intermediate node can exist on each path between node A and node B. The data processing unit 140 can obtain the shortest path between the node A and the node B using the number of intermediate nodes for each path. For example, the data processing unit 140 can determine that, among various paths between node A and node B, a path with a smaller number of intermediate nodes is a shorter path.

Alternatively, the data processing unit 140 obtains the shortest path between the node A and the node B by using the number of intermediate nodes for each path, and may reflect a type of mutual association for each connection line. That is, weights can be set differently for each category of mutual association, and the weights may also be applied to mutual association that exists for each path.

Equation 1 is an example of an equation for calculating the shortest path between nodes.

d_{i,j}^{w}=Σ_{w}_{ₛₜ}_{∈g}_{_{i→j}}_{^{w}}f(wₛₜ) [Equation 1]

Here, wₛₜ is a mutual association index between two nodes s and t, f is a weight transformation function, and g_{i→j}^{w} is the shortest path between two nodes i and j. The data processing unit 140 can determine a value of Equation 1 for each path, and select a path having the lowest value or the highest value as the shortest path.

Next, the clustering coefficient for each node can be computed by Equation 2 and Equation 3. Here, the clustering coefficient may be referred to as a grouping coefficient, and can mean a probability that a specific node and neighboring nodes are connected to each other or a connection density between the specific node and neighboring nodes.

tᵢ^{w}=½Σ_{j,h∈N}wᵢⱼwᵢₕwⱼₕ [Equation 2]

Here, tᵢ^{w} means the number of triangles in a graph created around each node i of the knowledge network, N is the total set of nodes in the knowledge network, wᵢⱼ is a mutual association index between nodes i and j, wᵢₕ is a mutual association index between nodes i and h, and wⱼₕ is a mutual association index between nodes j and h.

$${C}^{w} = \frac{1}{n} {\sum }_{i ∈ N} \frac{2 {t}_{i}^{w}}{{k}_{i} ( {k}_{i} - 1 )} \qquad [ Equation 3 ]$$

Here, C^{w} means the clustering coefficient, tᵢ^{w} is the number of triangles in the graph created around each node i of the knowledge network, and kᵢ means a degree of node i, that is, a value of the degree of connectivity of node i in the knowledge network.

Next, the centrality index for each node is an index of whether a specific node has the function of a hub, and can be expressed as a nodal degree D_{nodal} value, a betweenness centrality (BC) value, a nodal efficiency E_{nodal} value, etc. Here, the D_{nodal} value is a value of the degree of connectivity of each node in the knowledge network, that is, an index indicating how strong or weak node i has connectivity in the knowledge network, the E_{nodal} value is a value of a degree of efficiency of node i in the knowledge network, that is, a value expressed as the reciprocal of the shortest path of Equation 1, and is a value with higher efficiency as the path is shorter, and the BC value is an index indicating the number of times that node i becomes a shortcut in the path between nodes in the knowledge network.

First, the D_{nodal} value can be computed by Equation 4.

D_{nodal}(i)=Σ_{j∈N}wᵢⱼ [Equation 4]

Here, wᵢⱼ is a mutual association index between nodes i and j, and N is a total set of nodes in the knowledge network.

The E_{nodal} value can be calculated by Equation 5.

$${E}_{nodal ( i )} = {\sum }_{j ∈ N , j \neq i} \frac{1}{{d}_{i , j}^{w}} \qquad [ Equation 5 ]$$

Here, N is a total set of nodes of the knowledge network, and d^{W}_{i,j} is a Value Indicating the Shortest Path computed in Equation 1.

Next, betweenness centrality (BC) can be computed by Equation 6.

$$BC ( i ) = {\sum }_{{h , j ∈ N}_{h \neq j , h \neq i , j \neq i}} \frac{{g}_{hj} ( i )}{{g}_{hj}} \qquad [ Equation 6 ]$$

Here, gₕⱼ means the shortest distance between nodes h and j, and gₕⱼ(i) means the shortest distance between h and j passing through node i.

Next, when it is determined that a predetermined node has a function of a hub, the data processing unit 140 can classify the characteristics of the hub. In this case, the characteristics of the hub can be classified into a kinless hub, a connector hub, a provincial hub, etc. Here, the kinless hub means a hub with the most influential hub, that is, a hub connected to nodes in many modules, the connector hub means a hub that connects modules in the knowledge network, and the provincial hub means a hub that has a high influence mainly within the module. Here, the module can be a structural configuration group obtained by subdividing the entire knowledge network.

To this end, modularity in the knowledge network can be computed as in Equation 7. The modularity means the number of types of configuration modules in the entire knowledge network.

$${Q}^{w} = \frac{1}{{l}^{w}} {\sum }_{i , j ∈ N} [ {w}_{ij} - \frac{{k}_{i}^{w} {k}_{j}^{w}}{{l}^{w}} ] {σ}_{mi , mj} \qquad [ Equation 7 ]$$

Here, kᵢ^{W}=Σ_{j∈N}wᵢⱼ means the sum of weights at node i, and l_{W}=Σ_{i,j∈N}wᵢⱼ means the sum of weights. δ_{mi,mj} is the kronecker delta, 1 for mi=mj, and 0 for the rest.

Next, the participation coefficient (PC) of the knowledge network module can be computed as in Equation 8.

$${PC}_{i} = 1 - {\sum }_{m ∈ M} {[ \frac{{k}_{i}^{w} ( m )}{{k}_{i}^{w}} ]}^{2} \qquad [ Equation 8 ]$$

Here, M means a set of modules, kᵢ^{W}(m) means the number of connections between node i and all the other nodes in module m, and module m means a structural configuration group obtained by subdividing the entire knowledge network.

In addition, a z score (within-module degree) of the knowledge network module can be computed as in Equation 9.

$${z}_{i}^{w} = \frac{{k}_{i}^{w} ( {m}_{i} ) - {\overline{k}}^{w} ( {m}_{i} )}{{σ}_{k}^{w} ( {m}_{i} )} \qquad [ Equation 9 ]$$

Here, mᵢ means node i in module m, kᵢ^{W}(mᵢ) means the degree of connectivity in module m of node i, and k̅(mᵢ), σₖ^{W}(mᵢ) refer to the mean and standard deviation of the degree distribution of connectivity within module m, respectively.

Through the computation of the indexes in Equation 9 above, it is possible to distinguish whether each node is a hub or not within the module. For example, as in the following, when the Z score of the knowledge network module is 2.5 or higher, it can be determined as a hub.

1. within-module z-score≥2.5: hub

2. within-module z-score<2.5: not hub

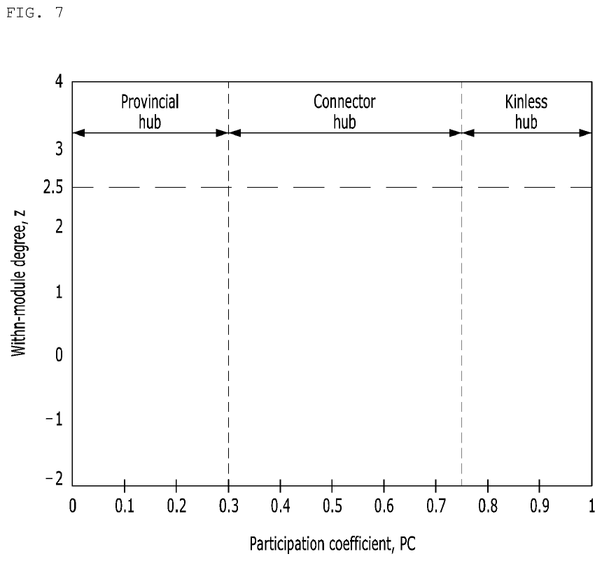

In addition, when it is determined that the node is a hub in the module, types of the hub can be classified as follows through the computation of the indexes in Equation 8, and FIG. 7 illustrates an example of classifying the types of the hub according to PC.

1. Provincial hub: PC≤0.30

2. Connector hub: 0.3<PC≤0.75

3. Kinless hub: PC>0.75

As described above, when the data processing unit 140 computes the graph theory index in step S240, the data refining unit 150 can generate a second knowledge network refined from the first knowledge network using the graph theory index (S250).

The second knowledge network is a network that is more simplified than the first knowledge network, and can be composed of only the nodes having high correlation in terms of the graph theory, among a plurality of nodes constituting the first knowledge network.

The nodes constituting the second knowledge network can be composed of nodes, of which the graph theory index computed in step S240 is equal to or greater than the reference value, among the plurality of nodes constituting the first knowledge network. For example, among a plurality of nodes constituting the first knowledge network, some nodes of which at least a part of an index value for the shortest path between nodes, an index value for the clustering coefficient for each node, and an index value for the centrality coefficient for each node is greater than or equal to a reference value, can be included in the second knowledge network. That is, the second knowledge network can be generated in such a way of deleting the nodes, of which at least a part of the index value for the shortest path between nodes, the index value for the clustering coefficient for each node, and the index value for the centrality coefficient for each node is less than the threshold value, among the plurality of nodes constituting the first knowledge network, and deleting the connections associated with the deleted nodes.

Here, the graph theory index compared to the reference value can be each of the index value for the shortest path between nodes, the index value for the clustering coefficient for each node, and the index value for the centrality coefficient for each node. Alternatively, the graph theory index compared to the reference value can be a value calculated by integrating at least two of the index value for the shortest path between nodes, the index value for the clustering coefficient for each node, and the index value for the centrality coefficient for each node.

According to an embodiment, at least one of the index value for the shortest path between nodes, the index value for the clustering coefficient for each node, and the index value for the centrality coefficient for each node can be computed as a standard score for each node, and the computed standard score can be compared with the threshold value.

Here, the standard score can be the z score, and the threshold value can mean 95% of significance. The Z score can be computed as in Equation 10.

$$z = \frac{X - mean ( x )}{SE ( x )} \qquad [ Equation 10 ]$$

Here, z is the z score, X is an index value of a predetermined graph theory index for a specific node in the first knowledge network, mean(x) is an average index value of predetermined graph theory indexes for at least some nodes in the first knowledge network, and SE(x) is a standard error of the index value of the graph theory index of at least some nodes in the first knowledge network. Here, it can be expressed as SE=σ/\sqrt{N}, where σ is the standard deviation, and n is the number of at least some nodes constituting the first knowledge network. According to an embodiment, the number of at least some nodes of the first knowledge network selected to determine the z-score can be 1000 nodes.

That is, the z score can be a value obtained by dividing the difference between the index value of the predetermined graph theory index for each of the nodes constituting the first knowledge network and the average index value of the predetermined graph theory index for the plurality of nodes constituting the first knowledge network by the standard error.

According to an embodiment, the z score can be computed through a permutation test. The permutation test can be performed in such a way of randomly mixing all the connection lines constituting the first knowledge network and then computing the z score for each node. In this case, the number of times of random mixing of the connection lines can be 1000 times or more.

The nodes constituting the second knowledge network may be some nodes which are extracted by using the index value for the hub characteristic for each node among the graph theory indexes computed in step S240 from among the plurality of nodes constituting the first knowledge network. That is, the node constituting the second knowledge network can be a node determined to be a hub within the module through the computation of the index of Equation 9, preferably a node classified as one of the kinless hub, the connector hub, and the provincial hub, more preferably a node classified as one of the kinless hub and the connector hub, and more preferably, a node classified as the kinless hub.

The data refining unit 150 can additionally remove unnecessary nodes of the first knowledge network in a process of analyzing a knowledge network. The data refining unit 150 can remove a node having one connection line together with a connection line of the corresponding node. This is because a node having only one connection line can be interpreted as a network node that does not conform to the concept of the multiomics network. In addition, the data refining unit 150 can remove a node having a clustering coefficient of 0 together with a connection line of the corresponding node. This is because, in the case of the node having the clustering coefficient value of 0, the node can be interpreted as a node that is unlikely to become a major hub node.

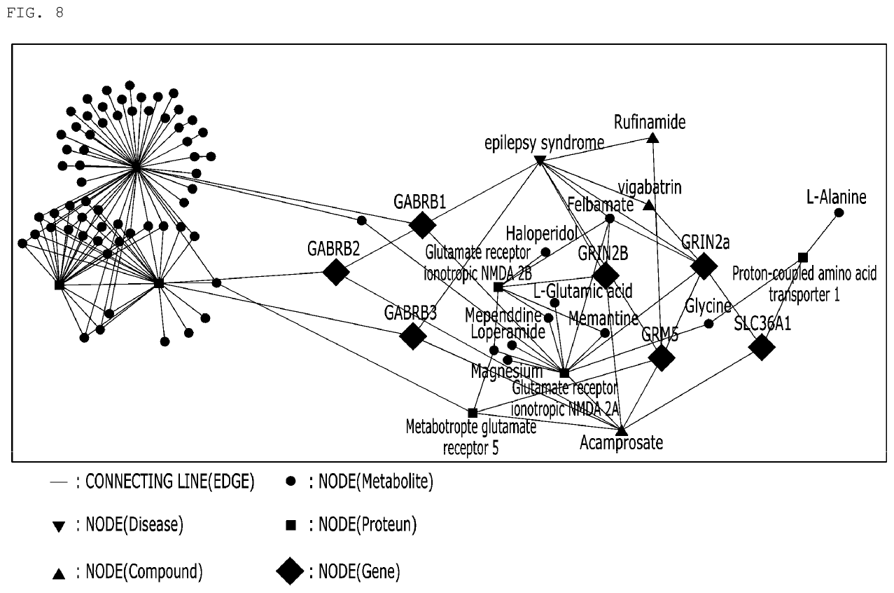

Next, the output unit 160 outputs the second knowledge network generated in step S250 (S260). The output unit 160 can be, for example, a display. FIG. 8 illustrates an example of the second knowledge network generated by using “epilepsy syndrome” as a search word according to an embodiment of the present invention. Referring to FIG. 8, it can be seen that the second knowledge network that is significantly simplified and refined compared to the first knowledge network of FIG. 6 can be obtained. In addition, referring to FIG. 8, it can be seen that biological entities within different omics levels associated with “epilepsy syndrome” and the mutual association between the biological entities can be intuitively obtained.

As described above, the apparatus for processing data 100 can generate the second knowledge network composed of only the nodes refined in relation to a predetermined search word, and accordingly, can easily determine a new drug candidate substance or a target of the new drug candidate substance.

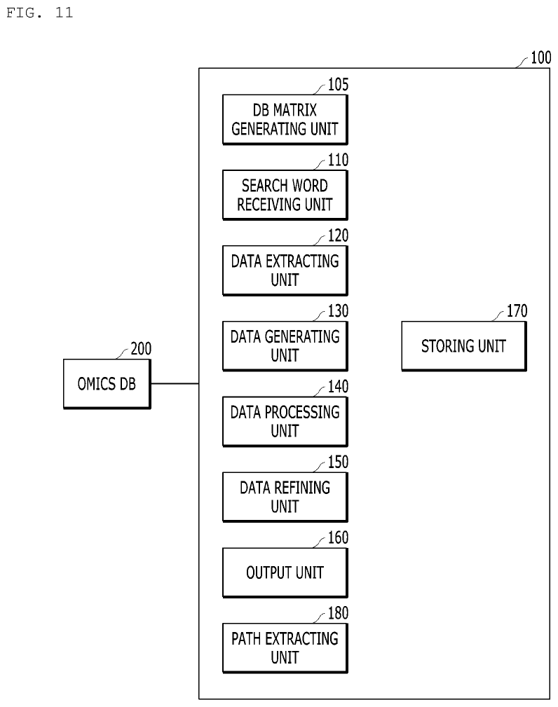

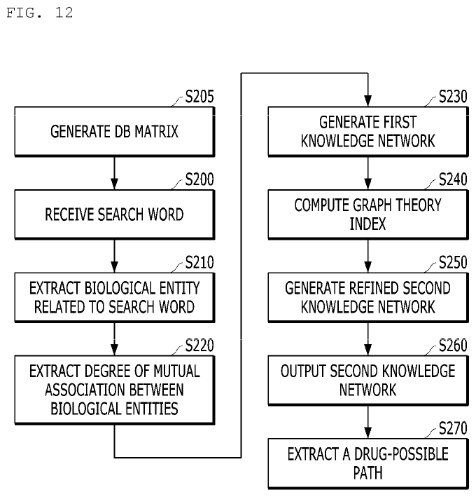

FIG. 11 is a block diagram of an apparatus for processing data for discovering a new drug candidate substance according to an additional embodiment and FIG. 12 illustrates a flowchart of a method for data processing to discover a new drug candidate substance by the apparatus for processing data according to an additional embodiment.

Referring to FIGS. 11 to 12, the apparatus for processing data 100 can further include a path extracting unit 180 for extracting a drug-possible path.

Here, the drug-possible path means a path to which a drug reacts or a path to which a drug acts, and can be used interchangeably with a drug reaction path or a drug action path. In this case, the drug-possible path can be displayed according to the degree of mutual association between biological entities in different omics levels, and can mean some connection paths in the second knowledge network generated in the present specification.

The path extracting unit 180 can extract a drug-possible path for determining a basic drug for deriving a new drug candidate substance by analyzing drug-disease node pairs existing in the second knowledge network (S270).

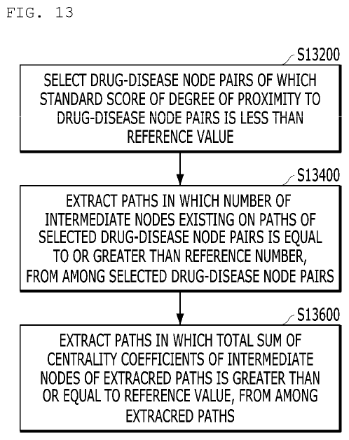

FIG. 13 illustrates a flowchart of how the apparatus for processing data searches for a drug-possible path according to an embodiment. The flowchart of FIG. 13 can represent sub-steps of step S270 of extracting the drug-possible path.

In step S13200, the path extracting unit 180 can select drug-disease node pairs of which the standard score (z-score) of the degree of proximity to each of the drug-disease node pairs existing in the second knowledge network is less than the reference value. The path extracting unit 180 can determine, from the second knowledge network, at least one drug-disease node pair that use a specific drug node and a disease node connected to the specific drug node through a connection line as a source node and a target node, respectively. According to an embodiment, the path extracting unit 180 can extract all the drug-disease pairs for the specific drug from the second knowledge network, and compute the standard score of the degree of proximity to each of the extracted drug-disease pairs. According to an embodiment, a standard score of the degree of proximity of a node pair (s, t) (s: source node (drug), t: target node (disease)) can be computed using Equation 11 below.

$$z ( s , t ) = \frac{d ( s , t ) - mean ( d ( s , T ) )}{S D ( d ( s , T ) )} \qquad [ Equation 11 ]$$

(s: source node, t: current target node, T: a set of target nodes, d(s, t): the shortest path (shortest distance) between source node s and current target node t, mean(d(s, T)): average of the shortest paths for node pairs consisting of source node s and target node set T, SD(d(s, T)): standard deviation of the shortest paths for node pairs consisting of source node s and target node set T, and z(s, t): standard score (z-score) of the degree of proximity of source node s to current target node t)

The path extracting unit 180 can select at least one drug-disease node pair of which the standard score (z-score) of the degree of proximity is less than a reference value. For example, if reliability is set to 90%, the reference value can be −1.645, if reliability is set to 95%, the reference value can be −1.960, and if reliability is set to 99%, the reference value can be determined to be −2.576.

In step S13400, the path extracting unit 180 can extract paths in which the number of intermediate nodes (i.e., the nodes that exist between the drug node and the disease node) existing on each of the paths is equal to or greater than the reference number among paths for pairs of which the degree of proximity of the drug-disease node pair selected in step S13200 is equal to or less than the reference value. For example, the path extracting unit 180 can extract paths of the drug-disease node pair, in which two or more intermediate nodes exist, from among the pairs extracted in step S13200.

In step S13600, the path extracting unit 180 can extract a path, in which a total sum of the centrality coefficients of the intermediate nodes is greater than or equal to the reference value from among paths in which the number of intermediate nodes extracted in step S13400 is equal to or greater than the reference number, as a drug-possible path. For example, the path extracting unit 180 can compute a total sum of centrality coefficients of intermediate nodes constituting the path for each of the paths in which the number of intermediate nodes extracted in step S13400 is greater than or equal to the reference number, and can extract paths having a higher total sum (e.g., within the top 1% of the distribution for the total sum of the centrality coefficients of intermediate nodes of the paths extracted in step S13400) as the drug-possible paths. With this configuration, the path extracting unit 180 can extract a drug-possible path that passes through a node having a high degree of concentration in the second knowledge network and increases the efficiency of a moving path.

The term ‘˜ unit’ used in this specification means (software or hardware components such as field-programmable gate array (FPGA) or ASIC, and the ‘˜ unit’ performs certain roles. However, the ‘˜ unit’ is not limited to software or hardware. The ‘˜ unit’ may be configured to be located in an addressable storage medium, or may be configured to reproduce one or more processors. Accordingly, as an example, the ‘˜ unit’ includes components such as software components, object-oriented software components, class components, and task components, processes, functions, attributes, procedures, subroutines, segments of program codes, drivers, firmware, microcode, circuits, data, databases, data structures, tables, arrays, and variables. Components and functions provided in the ‘˜ units’ can be combined into a smaller number of components and ‘˜ units’, or can be further separated into additional components and ‘˜ units’. In addition, components and ‘˜ units’ may be implemented to reproduce one or more CPUs in a device or a security multimedia card.

Meanwhile, the method for data processing described above can be implemented as computer-readable codes on a computer-readable recording medium. The computer-readable recording medium includes all the kinds of recording devices in which data readable by a computer system is stored. Examples of the computer-readable recording medium can include a ROM, a RAM, a CD-ROM, a magnetic tape, a floppy disk, an optical data storage device, etc. In addition, the computer-readable recording medium is distributed in a computer system connected through a network, so that a processor-readable code can be stored and executed in a distributed manner.

Descriptions are intended to provide exemplary configurations and actions for implementing the present invention. The technical idea of the present invention will include not only the embodiments described above, but also implementations that can be obtained by simply changing or modifying the above embodiments. In addition, the technical idea of the present invention will include implementations that can be achieved by easily changing or modifying the embodiments described above in the future.

1. A method for data processing to discover a new drug candidate substance performed by an apparatus for processing data, the method comprising:
generating a DB matrix composed of a selected biological entity and a selected type of mutual association degree from an omics DB;
receiving a search word;
extracting biological entities that belong to an omics level different from the search word and are related to the search word from the DB matrix;
extracting a degree of mutual association between the search word and the biological entities from the DB matrix;
generating a first knowledge network in which the search word and each of the biological entities are used as nodes and a plurality of nodes are connected using a connection line according to a degree of mutual association between the search word and the biological entities or a degree of mutual association between the biological entities;
computing a graph theory index for each of the plurality of nodes of the first knowledge network; and
generating a second knowledge network using some nodes selected using the graph theory index among the plurality of nodes of the first knowledge network,
wherein the search word includes at least one of a gene name, a protein name, a metabolite name, a symptom name, a disease name, a compound name, and a drug name,
the biological entities include at least one of genes, proteins, metabolites, symptoms, diseases, compounds, and drugs,
categories of the degree of mutual association include participate, covariate, regulate, associate, bind, upregulate, resemble, treat, downregulate, palliate, include, and express,
the graph theory index includes at least one of a shortest path between nodes, a clustering coefficient for each node, and a centrality coefficient for each node, for at least one of the plurality of nodes constituting the first knowledge network,
a weight of the connection line is set differently according to the category of the degree of mutual association indicated by the connection line, and the shortest path between nodes is calculated by reflecting the set weight,
in the generating of the second knowledge network,
the second knowledge network is generated by computing a standard score for at least one of the shortest path between nodes, the clustering coefficient for each node, and the centrality coefficient for each node and deleting a node whose standard score is less than a threshold value and a connection line of the node whose standard score is less than the threshold value, and
the standard score is a value obtained by dividing a difference between an index value of a predetermined graph theory index for each node constituting the first knowledge network and an average index value of the graph theory indexes for the plurality of nodes constituting the first knowledge network by a standard error, and
the DB matrix is generated such that the selected biological entities are arranged on a horizontal axis and a vertical axis, respectively, and the type of mutual association degree is displayed at a point where the horizontal axis and the vertical axis intersect.

2. The method of claim 1,
wherein the generating of the second knowledge network includes computing the standard score for each of the nodes of the first knowledge network after randomly shuffling all the connection lines constituting the first knowledge network, and the number of times of randomly shuffling is 1000 times or more.

3. The method of claim 1,
wherein the generating of the second knowledge network further includes
deleting a node having one connection line from among the nodes constituting the first knowledge network, and
deleting a node having a clustering coefficient of 0 from among the nodes constituting the first knowledge network.

4. The method of claim 1,
wherein the categories of the degree of mutual association further includes at least one of interact, cause, present, and localize.

5. The method of claim 1, further comprising:
extracting a drug-possible path from the second knowledge network,
wherein the extracting of the drug-possible path includes
selecting drug-disease node pairs whose standard score of a degree of proximity to each of the drug-disease nodes existing in the second knowledge network is less than a reference value,
extracting, from among paths for the selected drug-disease node pairs, paths in which the number of intermediate nodes existing in each of the paths is equal to or greater than a reference number, and
extracting, as the drug-possible path, a path in which a total sum of centrality coefficients of intermediate nodes of the extracted paths is equal to or greater than a reference value, from among the extracted paths.

6. A recording medium having recorded therein a program for causing the method performed according to claim 1 to be executed by a computer.
